(19) United States
(12) Patent Application Publication
(10) Pub. No.: US 2023/0191197 A1
(43) Pub. Date: Jun. 22, 2023

(54) SMART GLOVE

(71) Applicant: iFIT Inc., Logan, UT (US)

(72) Inventor: Darren C. Ashby, Richmond, UT (US)

(21) Appl. No.: 18/082,194

(22) Filed: Dec. 15, 2022

Related U.S. Application Data
(60) Provisional application No. 63/290,557, filed on Dec. 16, 2021.

(51) Int. Cl. A63B 24/00; A63B 71/14

(52) U.S. Cl. CPC A63B 24/0062; A63B 71/141; A63B 2220/836; A63B 2220/803; A63B 2024/0071; A63B 2220/30; A63B 2071/0625

(57) ABSTRACT

A method comprises detecting, by one or more processors of a wearable device, a tag identifier of a tag located on exercise equipment. The method also comprises selecting, from a memory of the wearable device, an algorithm from a plurality of stored algorithms responsive to the algorithm having a stored association with the tag identifier and receiving, from a sensor of the wearable device, movement data of a user performing an exercise associated with the exercise equipment. The method may also comprise executing the selected algorithm using the received movement data as input and determining, when the user has completed one repetition of the exercise based on the execution of the selected algorithm using the received movement data. The method may also comprise transmitting a record comprising repetition movement data that was received during the one repetition to a remote computing device.

This application claims the benefit of and priority to U.S. Provisional Patent Application No. 63/290,557, filed Dec. 16, 2021, which application is incorporated herein by reference in its entirety for all that it discloses.

### BACKGROUND

Tracking workout metrics provides many benefits but is generally time consuming and subject to human error. Some systems exist for automatically tracking workout metrics for cardio workouts performed on machines such as exercise bikes and treadmills, but these systems do not apply to strength training. Many exercisers engaging in strength training who wish to record their metrics write them in a notebook. They record things such as amount of weight lifted, number of repetitions per set, and number of sets. Manually writing this information can be a time-consuming and tedious process. Additionally, an exerciser's form may suffer when they are struggling to perform a target number of repetitions. Exercisers may also count some movements as repetitions that they should not.

Existing methods of tracking exercise movement may track movement but do not automatically track how much weight is being lifted. Thus, these methods cannot tell the difference between a movement performed holding a 5-pound weight and the same movement performed holding a 50-pound weight. Information of how much weight is being lifted must still be manually entered by an exerciser. Further, information on what exercise is being performed must be manually entered by the user. Entering this information is time-consuming and can distract an exerciser from a workout.

### SUMMARY OF THE INVENTION

One embodiment of the present disclosure relates to a method comprising detecting, by one or more processors of a wearable device, a tag identifier of a tag located on exercise equipment. The method may also comprise selecting, by the one or more processors from a memory of the wearable device, an algorithm from a plurality of stored algorithms responsive to the algorithm having a stored association with the tag identifier. The method may also comprise receiving, by the one or more processors from a sensor of the wearable device, movement data of a user performing an exercise associated with the exercise equipment. The method may also comprise executing, by the one or more processors, the selected algorithm using the received movement data as input. The method may also comprise determining, by the one or more processors, when the user has completed one repetition of the exercise based on the execution of the selected algorithm using the received movement data. The method may also comprise transmitting, by the one or more processors, a record comprising repetition movement data that was received during the one repetition to a remote computing device.

One embodiment of the present disclosure relates to a system comprising one or more processors, a memory, a plurality of communications interfaces, and one or more sensors, wherein the one or more processors detect a tag identifier of a tag located on exercise equipment. The one or more processors may also select, from the memory of the wearable device, an algorithm from a plurality of stored algorithms responsive to the algorithm having a stored association with the tag identifier. The one or more processors may also receive, from a sensor of the one or more sensors via a first communications interface of the plurality of communications interfaces, movement data of a user performing an exercise associated with the exercise equipment. The one or more processors may also execute the selected algorithm using the received movement data as input and determine when the user has completed one repetition of the exercise based on the execution of the selected algorithm using the received movement data. The one or more processors may also transmit, via a second communications interface of the plurality of communications interfaces, a record comprising repetition movement data that was received during the one repetition to a remote computing device.

### BRIEF DESCRIPTION OF THE DRAWINGS

The details of one or more implementations are set forth in the accompanying drawings and the description below. Other features, aspects, and advantages of the disclosure will become apparent from the description, the drawings, and the claims, in which:

FIG. 1A illustrates example wearable devices, in accordance with some embodiments of the present disclosure.

FIG. 1B is a block diagram of an exercise environment, in accordance with some embodiments of the present disclosure.

FIG. 2 is a block diagram of an example embodiment of a wearable device in communication with a remote computing device and exercise equipment via different communication interfaces, in accordance with some embodiments of the present disclosure.

FIG. 3 is a block diagram of an example embodiment of a database containing tag identifiers and algorithms, in accordance with some embodiments of the present disclosure.

FIG. 4 is a block diagram of an example embodiment of an algorithm using select indexes of a vector, in accordance with some embodiments of the present disclosure.

FIG. 5 is an example flow chart outlining a wearable device executing an algorithm to determine repetitions of an exercise from movement data, in accordance with some embodiments of the present disclosure.

FIG. 6 is an example user interface of a wearable device, in accordance with some embodiments of the present disclosure.

FIG. 7 illustrates an example embodiment, in accordance with some embodiments of the present disclosure.

FIG. 8 illustrates example sensor positions on a wearable device, in accordance with some embodiments of the present disclosure.

FIG. 9 is a flowchart of a method for performing an exercise activity, in accordance with at least one embodiment of the present disclosure.

### DETAILED DESCRIPTION

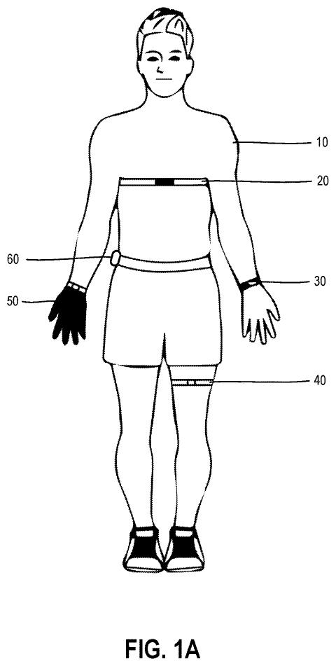

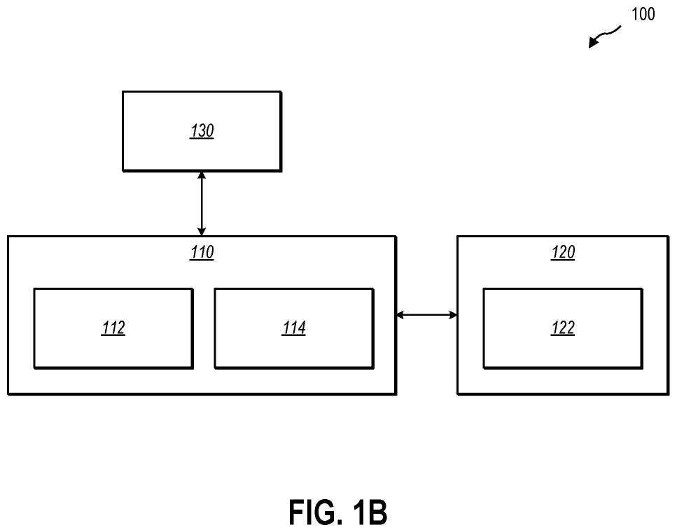

FIG. 1A illustrates example wearable devices. A user 10 may have a first wearable device 20 attached to a chest of the user 10. The user 10 may have a second wearable device 30 attached to a wrist of the user 10, such as a smart watch, an activity tracker, or other wrist-mounted second wearable device 30. The user 10 may have a third user device 40 attached to a thigh of the user 10. The user 10 may have a fourth wearable device 50 attached to a hand of the user 10. For example, the fourth wearable device 50 may include a glove, such as a weightlifting glove, a grip-increasing glove, a glove worn for warmth, any other type of glove, and combinations thereof. The user 10 may have a fifth wearable device 60 attached to a hip of the user. The user 10 may wear one or more of the wearable devices. As discussed herein, the wearable devices may include any type of wearable device.

In some embodiments, the wearable devices may include one or more sensors, such as accelerometers, gyroscopes, heart rate sensors, pulse oximeters, electrocardiograms (EKG), near-field communication (NFC) sensors, wireless communication antennas (e.g., Wi-Fi, Bluetooth, infrared), any other sensor, and combinations thereof. The sensors may be in communication with other elements of an exercise system using one or more communications interfaces. A communications interface may include any type of communications interface. For example, the communications interface may include a wired communication interface, a wireless communications interface, any other communications interface, and combinations thereof. In some embodiments, the wearable devices may include a plurality of communications interfaces. Different communications interfaces may be used to communicate with different elements of the exercise system. In some embodiments, each wearable device may include the same type of sensor. In some embodiments, each wearable device may include different types of sensors. In some embodiments, different wearable devices may include a combination of the same and different types of sensors, based on the location of the wearable device and the data to be collected by the wearable device.

The wearable devices may communicate with one another and/or a remote computing device. Movement data received from the wearable devices may be aggregated to track exercise performance such as repetitions. Each wearable device may be worn on a body part of the user. For example, a first wearable device on a user's chest may capture first movement data at the user's chest. A second wearable device on a user's hip may capture second movement data at the user's hip. When the user engages in pushups, the first wearable device may capture movement data at the user's chest and the second wearable device may capture movement data at the user's hip. The second wearable device may transmit the second movement data to the first wearable device to aggregate the movement data captured at a chest and a hip of a user at the first wearable device. The first wearable device may execute a first algorithm from a plurality of stored algorithms which takes as input movement data captured at the chest and the hip of the user. The first algorithm may be more accurate than a second algorithm which takes as input movement data from only a chest or only a hip of a user. The first wearable device may transmit a number of repetitions of the pushups obtained by the algorithm to a remote computing device. The wearable devices may be moved to other locations and/or body parts on the user's body. For example, the first wearable device 20 may prompt the user 10 using a visual/audible/tactile signal (e.g., an audible and/or visual signal) to move the first wearable device 20 to the hip of the user 10.

As discussed herein, the wearable devices may collect information from other exercise devices. For example, the fourth wearable device 50 may include a glove having an NFC sensor. A dumbbell may include an NFC ID chip (such as an RFID chip or other NFC ID chip). When the user picks up the dumbbell with the fourth wearable device 50, the wearable device 50 may recognize the dumbbell, including the data associated with the NFC ID chip (such as dumbbell type, dumbbell weight, dumbbell location). This may allow the user to have more detailed information regarding the dumbbell used to perform an exercise activity. This may provide the user with more detail regarding the exercise activity performed, thereby improving the exercise experience.

In the embodiment shown, the user 10 is wearing a single fourth wearable device 50 as a glove on his right hand. However, it should be understood that the user 10 may wear the fourth wearable device 50 on either hand. In some embodiments, the user 10 may wear the fourth wearable device 50 on both hands. For example, the user 10 may wear a first fourth wearable device 50 on the right hand and second fourth wearable device 50 on the left hand. This may allow the user to collect exercise information for both hands. For example, some exercise activities are single-handed, such as dumbbell bicep curls. Wearing the fourth wearable device 50 on both hands may allow the user to collect exercise information for exercise activities performed by both arms. As may be understood, the user 10 may wear any number of wearable devices on any portion of his body. For example, the user may wear the third wearable device 40 on either or both legs. The user 10 may wear the second wearable device 30 on either or both wrists. In this manner, the user 10 may wear an amount of wearable devices at locations that are targeted based on the exercise goals and/or desires of the user 10.

FIG. 1B is a block diagram of an exercise environment 100, in accordance with some embodiments of the present disclosure. The exercise environment 100 may include a wearable device 110. The wearable device 110 may be a smart watch, a smart bracelet, a fitness tracker, a smart ring, or any other wearable device. The wearable device 110 may include a processor 112 and a user interface 114. The exercise environment 100 may include a remote computing device 130. The exercise environment 100 may include exercise equipment 120. The exercise equipment 120 may be a dumbbell, a pair of dumbbells, a barbell, a kettlebell, a medicine ball, a resistance band, a mat, a pull-up bar, parallel bars, or any other exercise equipment. The exercise equipment 120 may also be any exercise machine including weights, pulleys, flywheels, or any other exercise equipment. The exercise equipment 120 may include a tag 122. The tag 122 may be RFID tags, barcodes, QR codes, or any tag capable of being scanned using near field communication.

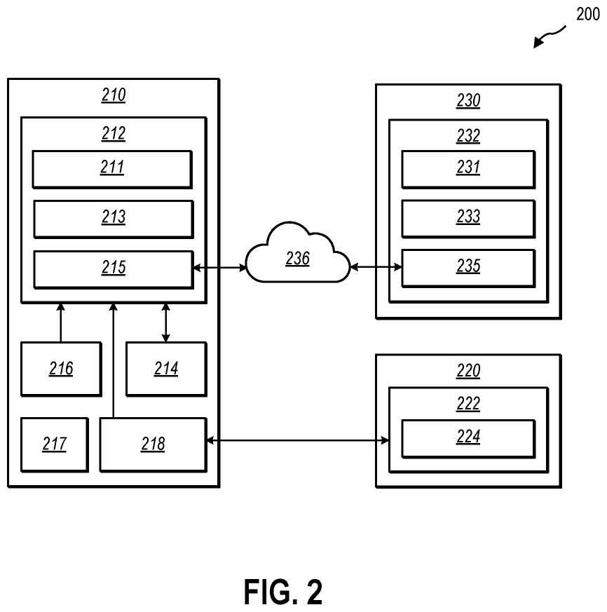

FIG. 2 is a block diagram of an exemplary embodiment of an exercise system 200 of a wearable device 210 in communication with a remote computing device 230 and exercise equipment 220 via different communication interfaces, in accordance with some embodiments of the present disclosure. The wearable device 210 may include a wearable device processing circuit 212, a user interface 214, a sensor 216, a battery 217, and a scanner 218. The wearable device processing circuit 212 may include a processor 211, a memory 213, and a network interface 215. The exercise equipment may include a tag 222. The tag 222 may include a tag identifier 224. The remote computing device 230 may include a remote device processing circuit 232. The remote computing device 230 may be a server. The processing circuit 232 may include a processor 231, a memory 233, and a network interface 235. The network interface 215 of the wearable device processing circuit 212 may communicate via a network 236 with the network interface 235 of the remote computing device processing circuit 232. The wearable device 210 may transmit information to the remote computing device 230. The wearable device 210 may transmit a record of user movement data to the remote computing device 230.

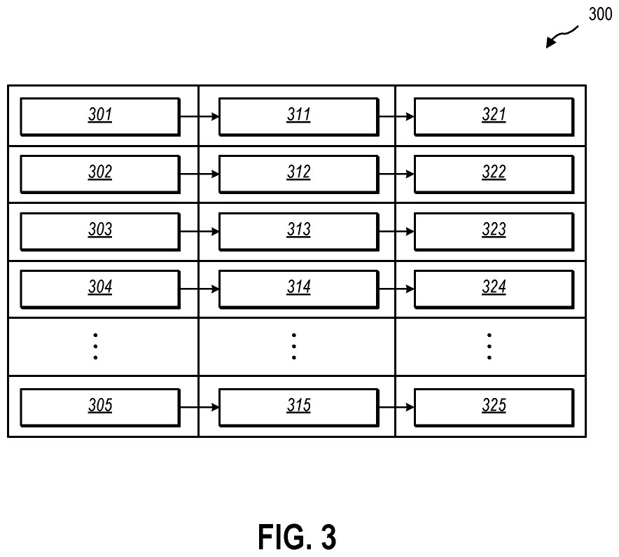

FIG. 3 is a block diagram of an exemplary embodiment of a database 300 containing tag identifiers and stored algorithms in accordance with some embodiments of the present disclosure. The database 300 may be stored in memory of a wearable device (e.g., memory 213 or memory 233). The database 300 may include a first tag identifier 301, a second tag identifier 302, a third tag identifier 303, a fourth tag identifier 304, and an nth tag identifier 305. The database 300 may include a first exercise 311, a second exercise 312, a third exercise 313, a fourth exercise 314, and an nth exercise 315. The database 300 may include a plurality of stored algorithms, including a first algorithm 321, a second algorithm 322, a third algorithm 323, a fourth algorithm 324, and an nth algorithm 325. The algorithms may have a stored association with the tag identifiers. For example, the first tag identifier 301 may be associated with the first exercise 311 and/or the first algorithm 321. The first algorithm 321 may be configured to count repetitions of the first exercise 311 based on movement data received by the wearable device. The wearable device may load the first algorithm 321 based on a stored association between the first tag identifier 301, the first exercise 311, and/or the first algorithm 321.

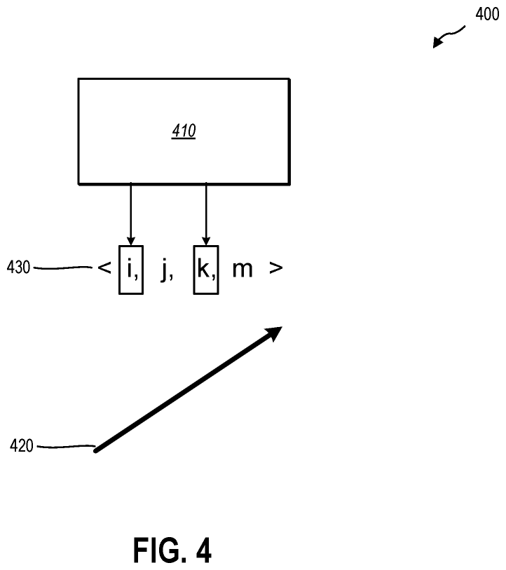

FIG. 4 is a block diagram of an exemplary embodiment of an exercise system 400 including an algorithm 410 executed by a processor (e.g., the processor 112) using select indexes of a vector 420 in accordance with some embodiments of the present disclosure. Movement data may be captured by a sensor of the wearable device such as an accelerometer or a gyroscope. The movement data may include vectors representing movement of the wearable device. A vector 420 may have index values at vector indexes 430. By executing the algorithm 410, the processor may select one or more vector indexes to use to determine when a user has completed a repetition of an exercise and key performance indicators (e.g., speed, acceleration, form, etc.) about the repetition. Different algorithms may select different sets of vector indexes. For example, the algorithm may be executed only upon the indexes of the vectors corresponding to movement along the vertical axis. Selecting only some of the indexes of the vector may have the advantage of simplifying the algorithm, reducing computing cost, and lowering bandwidth transmission requirements.

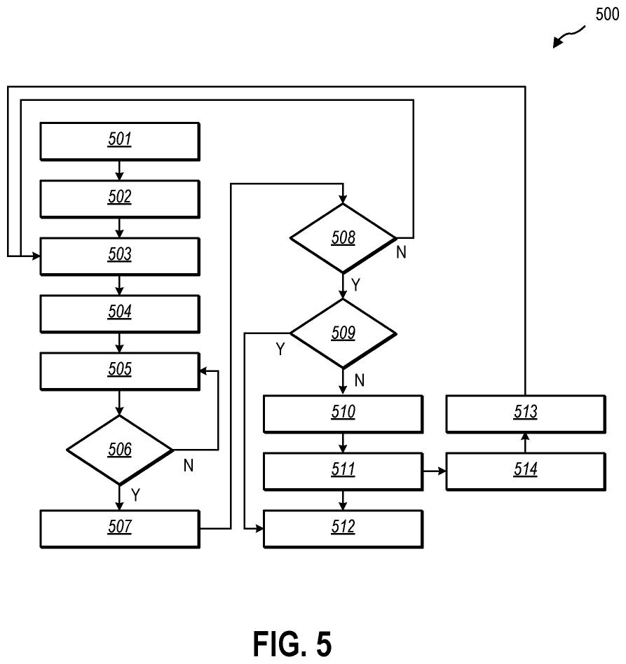

FIG. 5 is an example flow chart outlining a method 500 for executing an algorithm to determine repetitions of an exercise from movement data in accordance with some embodiments of the present disclosure. A processor (e.g., the processor 112 of the wearable device 110) may perform one or more of the operations of the method 500. The method 500 may include more or fewer operations and the operations may be performed in any order. At 501 a tag identifier is identified. At 502 an algorithm is selected corresponding to the tag identifier. At 503 movement data is received. At 504 index values is selected from movement data. At 505 the algorithm is executed on the selected index values. At 506 whether or not a repetition of the exercise has been completed is determined. At 507 an increment counter is maintained and/or updated. At 508 whether a number of repetitions exceeds a threshold is determined using repetition movement data. At 509 whether an exercise is the last exercise is determined. At 510 an identification of a next exercise is retrieved. At 511 an identification of the next exercise is presented. At 512 a record of the repetition movement data is generated. At 514 a tag identifier for the next exercise is identified. At 513 an algorithm corresponding to the tag identifier is selected.

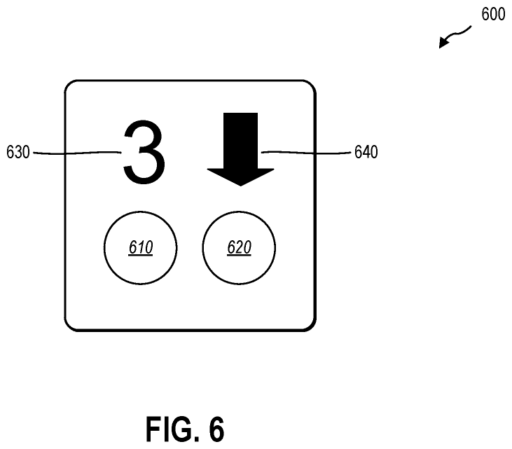

FIG. 6 is an example user interface 600 of a wearable device (e.g., wearable device 110) in accordance with some embodiments of the present disclosure. The user interface 600 may include a first button 610 and a second button 620. The user interface 600 may include a first indicator 630 and a second indicator 640. The user interface 600 may indicate any combination of the following: a number of repetitions of an exercise, an indication of whether the user should perform the exercise faster or slower, a time spent on the exercise, an indication of a next exercise, an indication of progress in a workout, and an estimated amount of calories burned. The user interface 600 may include a display. In some embodiments the user interface 600 may be a touchscreen display. The user interface 600 may include one or more user input interfaces. The user input interfaces may be buttons, switches, or areas on a display. User input may include an alteration to the sensitivity of one or more algorithms executed by the wearable device. Altering the sensitivity of the algorithms may include altering one or more weights associated with values in the movement data of the user. For example, a user may alter the sensitivity of an algorithm to multiply indexes of vectors of the movement data corresponding to movement along a vertical axis. The selected indexes of the vectors may be multiplied by a weight as the algorithm is executed on the selected indexes. This may allow the user to tune the sensitivity of the algorithm.

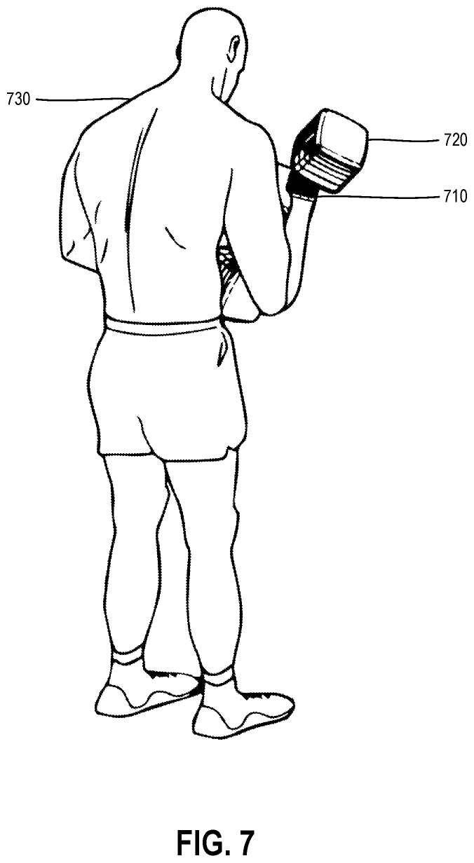

FIG. 7 illustrates an example embodiment. A user 730 may wear a wearable device 710 which counts repetitions of an exercise performed using exercise equipment 720. In the embodiment shown, the wearable device 710 is a glove and the exercise equipment 720 is a dumbbell held in a single hand by the user 730. The user 730 may scan a tag on the exercise equipment 720 using the wearable device 710. For example, when the user picks up the exercise equipment 720, the wearable device 710 may identify the exercise equipment 720, such as through an RFID sensor on the wearable device 710 and an RFID tag on the exercise equipment 720. In some embodiments, the RFID tag may be located on the handle of the exercise equipment 720. When the user places his hand on the handle, the RFID sensor on the wearable device 710 may identify the exercise equipment 720 using the RFID tag on the handle. In some embodiments, the RFID sensor may be located on the palm of the glove wearable device 710. In some embodiments, the RFID sensor may be located at any location of the wearable device 710

In some embodiments, the wearable device 710 may identify a tag identifier of the tag on the exercise equipment 720. In some embodiments, the wearable device 710 may determine an association between the tag identifier and an exercise and/or an algorithm in a database. For example, a particular unit of exercise equipment 720 may be associated with a particular exercise. In some embodiments, the tag of the exercise equipment 720 may include a weight associated with the exercise equipment 720. When the wearable device 710 identifies the exercise equipment 720, the wearable device 710 may identify the exercise and/or the algorithm based on a combination of the type of exercise equipment 720 and the weight of the exercise equipment 720. In some embodiments, the wearable device 710 may identify the exercise and/or the exercise equipment 720 based on an exercise activity scheduled to be performed during an exercise program. For example, an exercise program may include multiple exercise activities that are performed using the same exercise equipment 720, or the same type of exercise equipment 720 having different weights, the same exercise equipment 720 having variable weight (e.g., user-decided based on how he feels at the time of the exercise activity), any other type of exercise equipment 720, and combinations thereof. The wearable device 710 may identify the exercise and/or the algorithm based at least in part on the next-scheduled exercise activity in the exercise program. In some embodiments, an exercise activity may include multiple units of exercise equipment 720 and/or multiple types of wearable devices 710 may interact with or engage the exercise equipment 720. The wearable device 710 may determine the exercise and/or the algorithm based on information from multiple units of the exercise equipment 720 and/or information from multiple wearable devices 710.

The wearable device 710 may execute the algorithm on movement data received by the wearable device 710 and determine a number of repetitions of an exercise performed by the user 730. As discussed herein, an exercise controller may receive exercise information from other wearable devices. In some embodiments, the number of repetitions and/or the type of exercise device may be determined based on information from multiple wearable devices. The wearable device 710 may create a record of the number of repetitions of the exercise performed by the user 730 and transmit the record to a remote computing device.

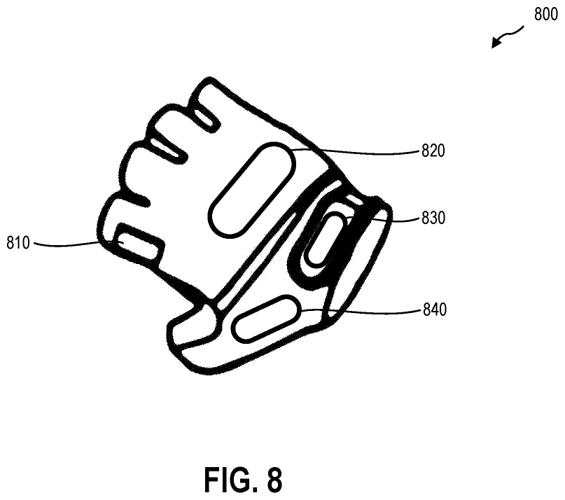

FIG. 8 illustrates an example wearable device 800 with example sensor attachment locations 810, 820, 830, and 840. Sensors may be attached to the wearable device 800 at 810 on a finger of the wearable device, at 820 on a back portion of the wearable device, at 830 on a wrist portion of the wearable device, and at 840 on a thumb portion of the wearable device. The wearable device 800 may have one or more sensors. Sensors of the wearable device 800 may be moved between different locations on the wearable device. Different algorithms may be used to track exercises based on the location of the one or more sensors. The wearable device 800 may prompt a user using a visual/audible/tactile signal to move the wearable device using an audible/visual/tactile signal to move the sensor from a first location to a second location. Although the wearable device is illustrated as a glove, other wearable devices may also have multiple attachment points for sensors.

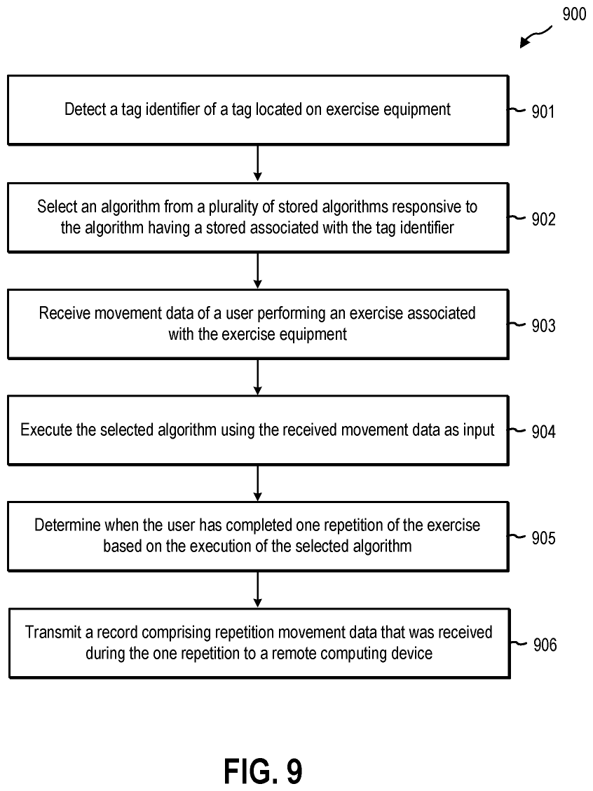

FIG. 9 is a flowchart of a method 900 for performing an exercise activity, according to at least one embodiment of the present disclosure. The method 900 may be implemented on one or more processors of a wearable device. Put another way, the acts of the method 900 may be performed by a processor on the wearable device. The method 900 may include detecting a tag identifier of a tag located on exercise equipment at 901. The tag identifier may be detected using any technology. For example, the tag identifier may be detected using an RFID sensor and an RFID chip. In some embodiments, the processor on the wearable device may select from a memory of the wearable device, an algorithm from a plurality of stored algorithms at 902. The selection may be responsive to the algorithm having a stored association with the tag identifier. A computing device may receive, from the processor on the wearable device, movement data of a user performing an exercise associated with the exercise equipment at 903. The processors on the wearable device may execute the selected algorithm using the received movement data as input at 904. The processors may then determine when the user has completed one repetition of the exercise based on the execution of the selected algorithm using the received movement data at 905. The processors may transmit a record comprising repetition movement data that was received during the one repetition to a remote computing device at 906.

### INDUSTRIAL APPLICABILITY

The system discussed herein allows a user to engage in exercise and gain all the benefits of tracking exercise metrics without having to document those metrics. A user can simply scan a tag on exercise equipment using the wearable device and engage in exercise without recording in a notebook or device what exercise is being performed or what weight is being lifted. As the user exercises, the wearable device tracks the movement of the user and from that movement determines what exercise is being performed, how many repetitions the user performs, and the form of the user, including how quickly the user is moving. For example, a user engaging in bicep curls can scan a tag on a 45-pound dumbbell to identify a tag identifier of the tag. The wearable device accesses a database in memory of the wearable device and load an algorithm for tracking bicep curls associated with the tag identifier. The tag identifier may be associated (e.g., have a stored association in a database) with other information, such as the weight of the dumbbell being 45 pounds. Then, when the user starts to exercise, the wearable device can track, using one or more sensors, the movement of the user engaging in bicep curls. The wearable device may execute the algorithm to count repetitions of the user's bicep curls with the 45-pound weight using the movement data. The wearable device may combine the weight of the dumbbell with the number of repetitions and calculate work performed, calories burned, or other useful metrics.

The wearable device can send a record (e.g., a file, document, table, listing, message, notification, etc.) indicating how many repetitions were performed to a remote computing device. Sending the record may include notifying the user of exercise information, such as on a display of the wearable device. The record may be sent after each repetition or after a set number of repetitions. The record may include one repetition or all the repetitions in a set. In some embodiments, the record may be cumulative, representing how many repetitions have been performed in a given set or workout. In some embodiments, the record of how many repetitions were performed may represent how many repetitions have been performed since the wearable device last sent a record of how many repetitions have been performed. This allows for a record to be kept of a user's exercise without requiring the user to document their own activity. This also gives the user valuable information about their form, including the speed at which the exercise is performed.

The system discussed herein may also allow a user to track their exercise performed on exercise equipment lacking sensors or transmitters. A user may place tags on existing exercise equipment and gain the benefits of recording their exercise metrics without having to manually document their exercise and without having to use specialized equipment with sensors for recording activity. This allows a user to record exercise on a variety of exercise equipment, including equipment that the user already owns. The user may customize the tag identifiers associated (e.g., via a stored association on a database) with tags according to the equipment on which the tags are placed. This is an improvement over existing systems for recording workout metrics using sensors built into equipment.

The functionality described herein is made possible by several technical improvements to tracking and recording exercise activity. For example, the wearable device may track much more information than can be easily transmitted from the wearable device (e.g., for a single repetition, a gyroscope that generates movement may generate thousands of vectors with many index values that may each occupy a portion of memory). To overcome this, the wearable device may receive vectors representing user movement, select vector indexes, and transmit only the select vector indexes. Alternatively, the wearable device may analyze the select vector indexes and transmit a record of the number of repetitions performed based on the select vector indexes. Transmitting either only the select vector indexes or a record of repetitions based on the select vector indexes may reduce the bandwidth transmission requirements of the wearable device. This improvement in bandwidth requirements may result in reduced computing load, longer battery life, the ability to analyze and transmit data in real time, and the ability to transmit other information from the wearable device.

To achieve the aforementioned technical advantages, a processor of a wearable device executes an algorithm on a portion of movement data received (e.g., received movement data) from a sensor of the wearable device. Executing the algorithm on received movement data may include selecting vector indexes of vectors in the received movement data for analysis. Executing the algorithm on received movement data may include determining when a repetition of the exercise has been completed. Determining when a repetition of the exercise has been completed may include calculating when a velocity of the wearable device reaches a certain value, calculating when the wearable device has traveled a predetermined threshold, such as a predetermined distance, calculating when an acceleration of the wearable device reaches a certain value, or any combination of the above. For example, a repetition of a bicep curl may be defined by an algorithm as lifting a dumbbell from a first position to a second position and back to the first position. The first position may be defined as a position where the velocity of the wearable device went from about zero to a positive value. The second position may be a predetermined height from the first position or it may be defined as a position above the first position where the velocity of the wearable device reaches zero. Returning to the first position to complete the repetition may be defined by the algorithm as traveling the distance between the first position and the second position in a downward direction or reaching a third position where the velocity of the wearable device reaches zero. Executing the algorithm on received movement data may include counting repetitions of an exercise. Executing the algorithm on received movement data may include counting sets of an exercise.

Additionally, the system discussed herein uses the tags to create workouts in real-time. The wearable device scans a tag on exercise equipment, identifies a tag identifier, and determines an algorithm associated with the tag identifier in a database. The algorithm is associated with an exercise to be performed using the exercise equipment. The wearable device receives movement data of the user and executes the algorithm on the movement data. The algorithm may output a number of repetitions of the exercise while the user is performing the exercise and an indication that the user has stopped performing repetitions of the exercise when the user stops performing the exercise. When the output of the algorithm indicates that the user has stopped performing the exercise, the wearable device may cease to execute the algorithm on the movement data. In some embodiments, the wearable device may receive second movement data and execute a general algorithm which determines which exercise the user is performing. The general algorithm may be an algorithm that is configured to determine which exercise is being performed by comparing movement data to a series of exercises, finding the closest match, and select an algorithm associated with the exercise which is the closest match. The selected algorithm then attempts to count repetitions of the exercise which is the closest match. If the selected algorithm is able to utilize an amount of the movement data satisfying a threshold in counting repetitions, the selected algorithm continues to execute on the movement data. If the selected algorithm is unable to utilize an amount of the movement data satisfying a threshold in counting repetitions, the selected algorithm will cease to execute on the movement data and the general algorithm will be used to determine which exercise is being performed.

The wearable device may, in response to determining which exercise the user is performing, load a second algorithm associated with a second exercise from the database. The wearable device may execute the second algorithm on the second movement data to determine a number of repetitions of the second exercise. In another embodiment, the wearable device may scan a third tag, identify a third tag identifier, and determine a third algorithm associated with the third tag identifier and a third exercise in the database. The process of determining that a user has ceased performing an exercise and begun another exercise and determining which exercise is being performed may be performed iteratively in order to combine several exercises into a workout. This has the advantage of allowing the user to create workouts in real time. The user is able to begin an arbitrary exercise without pausing to input what exercise has been chosen, and the system will record what exercise is being performed and how many repetitions the user performs. This means that the user is not restricted by a predetermined workout plan, but can create a workout as the user exercises. The user can create a workout in real time by performing several different exercises. The wearable device may record the exercises of the user. Exercises performed by the user in a continuous period of time may be termed a workout of the user.

The system may include a wearable device, a remote computing device, and exercise equipment. The wearable device includes a processor in communication with a user interface. The exercise equipment includes a tag. The wearable device communicates with the remote computing device and the exercise equipment. The wearable device may be a smart watch, a smart bracelet, a fitness tracker, a smart ring, or any other wearable device. The exercise equipment may be a dumbbell, a pair of dumbbells, a barbell, a kettlebell, a medicine ball, a resistance band, a mat, a pull-up bar, parallel bars, or any other exercise equipment. The exercise equipment may also be any exercise machine including weights, pulleys, flywheels, or any other exercise equipment.

The tag has a tag identifier. The scanner of the wearable device scans the tag and identifies the tag identifier. In some embodiments, the tag identifier may be unique to the tag. In some embodiments the tag identifier may be shared among multiple tags on identical or similar pieces of exercise equipment. In some embodiments, the tag identifier may be customized by the user.

The wearable device scans the tag and receives the tag identifier using a communications interface. The communications interface may be a scanner of the wearable device. The scanner of the wearable device may a Radio Frequency Identification (RFID) scanner, a laser scanner, a near field communication reader, or any other type of scanner. The tags may be RFID tags, barcodes, QR codes, or any tag capable of being scanned using near field communication. The tag may be a passive object without electrical power. The wearable device may scan the tag and receive information including the tag identifier. The wearable device may, based on associations with the tag identifier in a database, determine an identity of the exercise equipment, one or more parameters of the workout equipment (e.g., dimensions, weight, configuration, etc.), an exercise associated with the tag, and/or an algorithm associated with the exercise. In some embodiments, the wearable device may determine additional information associated with the tag identifier in the database including an identity of the exercise equipment the tag is on, an exercise associated with the tag, and an algorithm associated with the exercise.

In some embodiments the exercise equipment may be identified by a camera. The camera may be part of a computing device. The computing device may be the wearable device. A user may indicate a choice of exercise equipment. The indication may include a user picking up the chosen exercise equipment, pointing at the chosen exercise equipment, or touching the chosen exercise equipment. The camera may receive the indication of the choice of exercise equipment by capturing a picture of the user indicating the chosen exercise equipment. A processor may execute a gesture recognition algorithm to determine that an indication of chosen exercise equipment is being made by the user. For example, the camera may capture an image of a user picking up a dumbbell and the processor may execute a gesture recognition algorithm on the picture to determine that the user is picking up the dumbbell. In some embodiments, the camera may capture a picture of the exercise equipment. A processor of the computing device may execute an object recognition algorithm and/or an optical character recognition (OCR) algorithm to identify the exercise equipment. For example, the camera may capture a picture of a dumbbell and the processor may execute an object recognition algorithm to determine that the picture represents a dumbbell and execute an OCR algorithm to identify markings on the dumbbell indicating the weight of the dumbbell. Using the identification of the dumbbell, the computing device may identify an algorithm associated with an exercise to be performed using the exercise equipment in a database (e.g., the computing device may query the database using the identification of the dumbbell as an input). In some embodiments, the computing device may capture movement data of a user using motion sensors or the camera. The camera may capture movement data by capturing a video or a series of pictures of the user performing the exercise. The processor of the computing device may execute a motion capture algorithm to determine movement of the user from the video or the series of pictures of the user performing the exercise. The processor of the computing device may execute an algorithm as disclosed herein for counting repetitions of the exercise.

In some embodiments, the camera may identify a tag on or near the exercise equipment. The tag may have a tag identifier. The tag identifier may be written, printed, or stamped on the tag. The tag identifier may be otherwise indicated on the tag. The camera may capture an image of the tag. The processor of the computing device may execute an OCR algorithm to identify the tag identifier. The processor of the computing device may identify the exercise equipment based on an association between the tag identifier and the exercise equipment in a database.

The tag may be used to represent a variety of things based on the relationship between the tag identifier of the tag and other elements in the database. For example, the tag identifier may be associated in the database with one exercise or with multiple exercises. The location of the tag does not constrain the relationship in the database between the tag identifier and exercises. The tag may be placed on the exercise equipment at or near the place where the user grips or contacts the exercise equipment. The tag may be placed on the exercise equipment at a convenient location for scanning the tag or at any other place on the exercise equipment. The exercise equipment may include more than one tag. For example, multiple tags may be placed on one piece of exercise equipment wherein each tag has a tag identifier associated with a different exercise to be performed using the exercise equipment. The tag may represent more than one piece of exercise equipment. For example, one tag may be placed on a dumbbell to represent that the user will use a pair of dumbbells including the dumbbell with the tag. In this example, the tag on the dumbbell has a tag identifier which is associated with one or more algorithms associated with one or more exercises that are to be performed using a pair of dumbbells. The tag identifier may also be associated with an exercise equipment identifier which identifies the pair of dumbbells.

The tag may also represent a sequence of exercises or a routine. For example, a tag on a mat may represent a series of exercises or stretches to be performed on the mat. In this example, the tag identifier of the tag may be associated in the database with a workout plan containing a series of exercises. The wearable device may load algorithms associated with the exercises in the workout plan, receive movement data of a user performing the series of exercises, and execute the algorithms on the user data. In some embodiments, the wearable device may automatically detect when the user transitions from one exercise to another as discussed herein. In other embodiments, the wearable device may receive input from the user indicating that the user is transitioning from one exercise to another. In yet other embodiments, the wearable device may determine, based on a number of repetitions of each exercise exceeding a predetermined threshold, such as a predetermined number of repetitions, that it is time to transition to another exercise and prompt the user with an audible, visual, and/or tactile signal at the wearable device. In further embodiments, the user may scan a tag with a tag identifier associated in the database with transitioning from one exercise to another.

In some embodiments the exercise equipment may include a tag on pieces of the exercise equipment that denote different exercises to be performed with the exercise equipment, different settings of the exercise equipment, or different weights associated with the exercise equipment. For example, a medicine ball may have tags on the medicine ball that denote various exercises to be performed with the medicine ball. The wearable device may scan more than one tag for a particular exercise. For example, if the exercise equipment is a squat rack, the wearable device may scan a tag on a barbell and tags on various weights that may be added to the barbell in order to record the total weight involved in the exercise.

The wearable device may access a third-party database. This grants the advantage of being able to access equipment data beyond what is in the database associated with the wearable device. For example, specialized exercise equipment may not have associated data in the database associated with the wearable device. However, a third-party database may contain data pertaining to the specialized exercise equipment. The wearable device may access a second database based on an association between a tag identifier and the second database. For example, a user may scan a tag on exercise equipment in the user's home with a tag identifier associated with a first database. The wearable device may identify the first database based on the tag identifier and access the first database. The user may scan a second tag with a second tag identifier on second exercise equipment in a gym associated with a second database containing equipment data pertaining to equipment in the gym. The wearable device may identify the second database based on the second tag identifier. The second database may be located on a server associated with the gym. The second database may also be downloaded to the wearable device via one or more communications interfaces of the wearable device. The second tag identifier of the second tag may identify the second database, be associated in the first database with the second database, or be otherwise associated with the second database. This allows the wearable device to scan tags and determine algorithms and exercises associated with their tag identifiers on or near a wide variety of exercise equipment in a variety of different locations.

The wearable device may communicate with exercise equipment having built-in sensors. The tag may be placed on workout equipment having built-in sensors. The tag identifier may be associated in the database with exercises to be performed using the exercise equipment and a network identifier of the exercise equipment. The wearable device may communicate with the exercise equipment through the network. The wearable device may receive information from the exercise equipment including performance metrics such as output, energy exerted, or pace. For example, the wearable device may receive from a rowing machine a pace of a user such as five hundred meters every two minutes and an amount of energy exerted by a user such as 10 kilojoules per minute or 100 kilojoules in total. The wearable device may transmit movement data received from the one or more sensors of the wearable device to the exercise equipment. For example, the tag may be placed on a stationary bike having one or more built-in sensors. The wearable device may identify a tag identifier of the tag associated with the stationary bike and a network identifier of the stationary bike. The network identifier may be an IP address, MAC address, URL, or other identifier which allows the wearable device to communicate with the stationary bike via a network such as the internet or a local wireless network. The wearable device may communicate with the stationary bike through the network. The stationary bike may determine performance metrics of the user by built-in sensors of the stationary bike. The bike may measure the rotation of a flywheel of the bike and the resistance applied to the flywheel to calculate a speed of the user's pedaling and an amount of energy expended. The stationary bike may transmit the performance metrics to the wearable device through the network. In some embodiments, the wearable device may transmit movement data of the user received by the one or more sensors of the wearable device to the stationary bike. The wearable device may combine the performance metrics from the stationary bike with the movement data of the user to determine when the user was on a seat of the stationary bike and when the user was standing on pedals of the stationary bike. The wearable device may annotate the performance metrics with information on whether the user was on the seat or standing on the pedals when the performance metrics were captured. The wearable device may modify the performance metrics based on when the user was on the seat of the stationary bike and when the user was standing on the pedals of the stationary bike. When using a stationary bike, the user can stand on the pedals and use the weight of their body to turn the pedals. This may result in the sensors of the exercise bike reading greater energy exerted than is actually exerted by the user. The wearable device may apply a weighting factor to the performance metrics captured while the user was standing on the pedals. In some embodiments the weighting factor may be based on the user's weight. This may result in more accurate estimates of energy exerted and calories burned.

In some embodiments, the wearable device may employ algorithms configured to take as input movement data captured from different parts of the body. The wearable device may be attached to a user at the user's wrist, finger, upper arm, thigh, hip, ankle, or at any other position for tracking exercise movement. The wearable device may be moved from a first position on the user's body to a second position on the user's body based on a first movement being more effectively measured by a sensor at the first position and a second movement being more effectively measured by a sensor at the second position. For example, a bicep curl may be more effectively measured by a sensor on the user's wrist while a squat may be more effectively measured by a sensor on the user's hip. An algorithm for tracking repetitions of a squat may include selecting indexes of vectors of the movement data corresponding to movement along a vertical axis, measuring a first position of the wearable device when the user is standing, observing when the wearable device is lowered beneath the first position, calculating when the velocity of the wearable device reaches zero at a second position beneath the first position, and observing when the wearable device returns to the first position. The squat algorithm in this example may be as follows:

1. Select index 3 of vectors of movement data.

2. At t=0, set height to 0.

3. When height<0 and velocity=0, set halfrep to 1

4. When height=0 and velocity=0, set halfrep to 2.

An example of a bicep curl algorithm may be as follows:

1. Select index 3 of vectors of movement data.

2. At t=0, set height to 0.

3. When height>0 and velocity=0, set halfrep to 1.

4. When height=0 and velocity=0, set halfrep to 2.

The wearable device may prompt a user with an audible, visual, and/or tactile signal at the wearable device to move the wearable device from the first position to the second position (e.g., from the person's wrist to the person's hip) in response to the user scanning a tag having a tag identifier associated with an algorithm that takes as input movement data captured from the second position. In some embodiments the user interface of the wearable device may allow the user to indicate whether the sensor has been moved. The wearable device may, in response to receiving input from the user that the wearable device has been moved to the second position, use a second algorithm that takes as input movement data captured at the second position. In response to not receiving input from the user that the wearable device has been moved to the second position after prompting the user to move the wearable device to the second position, the wearable device may use a first algorithm that takes as input movement data captured at the first position.

In some embodiments the user may wear a first wearable device and a second wearable device. The first wearable device may be attached to the user at a first location corresponding to a first algorithm configured to receive as input movement data captured at the first location. The second wearable device may be attached to the user at a second location corresponding to a second algorithm configured to receive as input movement data captured at the second location. The first or second wearable device may scan a first tag on first exercise equipment, the first tag having a first tag identifier associated with the first algorithm. The first wearable device may send an indication to the second wearable device that the exercise is associated with an algorithm configured to receive as input movement data captured at the first location. The first wearable device may receive from a sensor of the first wearable device first movement data of a user performing an exercise associated with the first tag identifier and the first algorithm. The second wearable device may receive from a sensor of the second wearable device second movement data of a user performing the exercise associated with the first tag identifier and the first algorithm. The first wearable device may execute, using one or more processors of the first wearable device, the first algorithm on the first movement device to determine a number of repetitions of the exercise. The second wearable device may abstain from executing an algorithm on the second movement data. The first wearable device may transmit the number of repetitions to a remote computing device.

The remote computing device may be a server, computer, smartphone, or any other computing device. The remote computing device communicates with the wearable device over a network. The network may be a local area network (LAN), wide area network (WAN), the Internet, or any other network. The remote computing device includes a processing circuit. The processing circuit of the remote computing device includes a processor, a memory, and a network interface. The wearable device includes a processing circuit. The processing circuit of the wearable device includes a processor, a memory, and a network interface. The network interface of the wearable device may communicate with the network interface of the remote computing device through the network.

The wearable device may include a user interface, a sensor, a battery and a scanner. The sensor may be an accelerometer, a gyroscope, or any other sensor for measuring movement. In some embodiments the wearable device may include more than one sensor. In some embodiments the system may include multiple wearable devices (e.g., a user performing an exercise may wear multiple wearable devices). In some embodiments, the multiple wearable devices may communicate with each other through the network and/or via a local connection using one or more communications interfaces. The multiple wearable devices may communicate using Bluetooth®, ANT+®, Wifi®, or any other communication protocol. A first wearable device may receive data from the other wearable devices of the multiple wearable devices. This may allow for more accurate movement data to be captured. For example, a first wearable device on a user's chest may capture first movement data at the user's chest. A second wearable device on a user's hip may capture second movement data at the user's hip. When the user engages in pushups, the first wearable device may capture movement data at the user's chest and the second wearable device may capture movement data at the user's hip. The second wearable device may transmit the second movement data to the first wearable device to aggregate the movement data captured at a chest and a hip of a user at the first wearable device. The first wearable device may execute a first algorithm which takes as input movement data captured at the chest and the hip of the user. The first algorithm may be more accurate than a second algorithm which takes as input movement data from only a chest or only a hip of a user. The first wearable device may transmit a number of repetitions of the pushups obtained by the algorithm to a remote computing device. In some embodiments, the multiple wearable devices may each transmit their captured movement data to the remote computing device without transmitting their movement data to a first wearable device of the multiple wearable devices.

The wearable device receives movement data from the sensor of the wearable device. For example, an accelerometer may measure acceleration of the wearable device during a period of time. The wearable device may calculate, from the acceleration of the wearable device, the velocity and position of the wearable device during the period of time. The wearable device may execute an algorithm on the position, velocity, and/or the acceleration of the wearable device. The algorithm may be configured to receive as input the position, velocity, and/or acceleration of the wearable device and determine one or more parameters based on movements of a user. For example, the wearable device may be placed on the user's wrist, allowing the algorithm to use the movement of the wearable device as the movement of the user's wrist when the user is performing bicep curls. The algorithm may be configured to determine when the user's wrist moves from a first position to a second position and then back to the first position. The first position may be the bottom of the curl and the second position may be at the top of the curl. The algorithm may iterate a counter each time it determines that the wrist of the user has moved from the first position to the second position and back to the first position. The counter may represent a number of repetitions of the bicep curl.

The wearable device may select an algorithm configured to receive movement data from the location of the wearable device based on the tag identifier of the tag. For example, if a user is performing bicep curls, the user may scan a tag on a dumbbell having a tag identifier associated in a database with bicep curls and an algorithm configured to receive movement data of a user's wrist and output repetitions of bicep curls.

The wearable device communicates with the remote computing device using one or more communications interfaces via a network. The one or more communications interfaces may include an antenna of the wearable device. The one or more communications interfaces may include the network interfaces of the wearable device and the remote computing device. The remote computing device may include workout information received from devices other than the wearable device. The remote computing device may be a web-based service for improving a user's fitness and wellbeing. The remote computing device may contain historic workout information of a user. The remote computing device may, based on historical workout information and current workout data, customize workouts in real-time as discussed herein and transmit the customized workouts to the wearable device. The wearable device may lack sufficient memory or processing power to store a large amount of data or process in real-time a large amount of data. The communications interface may allow data to be stored or processed on the remote computing device and transmitted to the wearable device. Additionally, the remote computing device may be too heavy or bulky to be attached to a user. The communications interface may allow the wearable device to be attached to the user and transmit movement data of the user to the remote computing device.

The wearable device transmits data to the remote computing device. The wearable device may transmit a tag identifier of a tag to the remote computing device. The wearable device may transmit an algorithm and/or exercise associated with the tag identifier to the remote computing device. The wearable device may transmit movement data of a user to the remote computing device. The wearable device may transmit a signal to the remote computing device to increase a count of repetitions of an exercise. The wearable device may record a number of repetitions of an exercise and transmit the record of the number of repetitions of the exercise to the remote computing device.

In some embodiments the wearable device may transmit additional data about a workout to the remote computing device such as performance parameters. For example, the wearable device may calculate an estimated calorie burn and transmit the estimated calorie burn to the remote computing device. The wearable device may calculate the estimated calorie burn based on information about the exercise equipment in the database and a number of repetitions of an exercise performed by the user. For example, a user may scan a tag on a dumbbell having a tag identifier associated in a database with an algorithm associated with bicep curls and a weight of the dumbbell. The wearable device may receive movement data from a sensor of the wearable device and execute the algorithm associated with the tag identifier on the movement data to determine a number of repetitions of the bicep curl as disclosed herein. The wearable device may estimate a calorie burn based on the weight of the dumbbell and the number of repetitions of the bicep curl. The wearable device may calculate the amount of physical work performed by the user in lifting and lowering the dumbbell over the number of repetitions and multiply it by a scaling factor based on the user's weight to obtain an estimated calorie burn. Other performance parameters may include a length of workout, work performed during a workout, average heart rate during a workout, and total time spent exercising during the workout. The remote computing device may transmit these performance parameters, among others, to the remote computing device.

A database contains multiple tag identifiers, multiple exercises, and/or multiple algorithms. The database may include any number of tag identifiers, exercises, and algorithms. Each of the tag identifiers is associated with an exercise. Each of the tag identifiers may be associated with exercise equipment. Each of the exercises is associated with an algorithm. In some embodiments, each of the tag identifiers may be associated with an algorithm. In some embodiments, some tag identifiers may be associated with more than one exercise and/or algorithm. For example, a tag identifier may be associated with a dumbbell and multiple exercises which may be performed using the dumbbell, and multiple algorithms configured to determine repetitions of the multiple exercises. The database may be located in the memory of the wearable device and/or in the memory of the remote computing device.

In some embodiments, the wearable device may determine, based on movement data of a user, which exercise is being performed. The wearable device may access the database and determine an algorithm associated with the exercise. The wearable device may use the algorithm to analyze the movement data of the exercise. The wearable device may determine when the user begins a second exercise. The wearable device may access the database and determine a second algorithm associated with the second exercise. The wearable device may use the algorithm to analyze the movement data of the second exercise.

Movement data captured by the wearable device may include vectors representing position, velocity, and/or acceleration. The movement data may be captured by a sensor of the wearable device such as an accelerometer or a gyroscope. In the case of the sensor being a gyroscope, the vectors of the movement data are gyroscopic vectors. Each vector may include various indexes corresponding to different aspects of the vector. An algorithm may select one or more indexes of the vector as input for the algorithm. In some embodiments the algorithm may select vector indexes which are necessary for analyzing form of a user and counting repetitions of an exercise and not select vector indexes which are not necessary for analyzing form of a user and counting repetitions of an exercise. For example, a gyroscope of a wearable device may capture gyroscopic vectors of the movement data representing movement of the wearable device in three dimensions. An algorithm may select indexes of the gyroscopic vectors corresponding to movement along a vertical axis in one dimension. The algorithm may select the indexes of the gyroscopic vectors corresponding to movement along the vertical axis based on a tag identifier of a scanned tag or the algorithm which is associated with the tag identifier. The algorithm may be executed only upon the indexes of the gyroscopic vectors corresponding to movement along the vertical axis. Different algorithms may select different sets of vector indexes. Selecting only some of the indexes of the vector may have the advantage of simplifying the algorithm, reducing computing cost, and lowering bandwidth transmission requirements. In some embodiments the algorithm may select a set of vector indexes based on a tag identifier scanned by the wearable device. In some embodiments the algorithm may select a set of vector indexes based on an exercise associated with the tag identifier and the algorithm.

In some embodiments the algorithm may select a first set of vector indexes, determine that the first set of vector indexes does not correspond to an exercise corresponding to the algorithm, and select a second set of vector indexes. The algorithm may determine that the first set of vector indexes does not correspond to the exercise corresponding to the algorithm by comparing the first set of vector indexes to an expected set of vector indexes. For example, a user may scan a tag on a dumbbell having a tag identifier associated in a database with the exercise of bicep curls and an algorithm for determining repetitions of bicep curls. The algorithm may select vector indexes of vectors of movement data of the user corresponding to movement along a y-axis. A set of expected vector indexes may indicate that during bicep curls, vector indexes corresponding to movement along a z-axis increase and decrease while vector indexes corresponding to movement along the y-axis do not change. The algorithm may compare the vector indexes corresponding to movement along the y-axis to the set of expected vector indexes and determine the difference between them. Based on the difference, the algorithm may select vector indexes corresponding to movement along the z-axis. The algorithm may compare the vector indexes corresponding to movement along the z-axis and determine the difference between them. Based on the difference, the algorithm may continue to execute on the vector indexes corresponding to movement along the z-axis. The algorithm may perform calculations upon the vector indexes corresponding to movement along the z-axis while ignoring or discarding other vector indexes of the movement data. The algorithm may determine, based on its calculations, a number of repetitions of the exercise. Limiting the calculations of the algorithm may allow the algorithm to determine a number of repetitions in real-time.

In some embodiments the algorithm may execute with consecutive vectors as input. Consecutive vectors may be vectors of movement data which were captured consecutively. The wearable device may compare consecutive vectors and/or data resulting from the algorithm executing with consecutive vectors as input. In some embodiments, the wearable device may compare, using the algorithm, consecutive vectors to identify a difference in values between the consecutive vectors. The wearable device may determine, using the algorithm, that the user has completed one repetition based on the difference between the consecutive vectors. The wearable device, using the algorithm, may compare the difference between the consecutive vectors to a repetition policy which defines what constitutes a repetition of the exercise. The repetition policy may be a series of rules which dictate when the algorithm determines that a repetition of the exercise has been performed. For example, a repetition policy for the exercise of bicep curls may dictate that a wrist of a user holding a dumbbell must move from a first lower position to a second higher position and then back to the first lower position. The repetition policy may also depend upon a velocity or acceleration. For example, the repetition policy for bicep curls in the previous example may dictate that a repetition has been completed only when the wrist of the user has moved back to the first lower position and has a velocity of zero. As another example, a repetition policy for bicep curls may identify y-values of a position of the wearable device corresponding to a position of the wearable device along a vertical axis. The repetition policy may count one repetition as when the y-values of the position of the wearable device change from increasing to decreasing. The repetition policy may count several repetitions of the same exercise.

In some embodiments the algorithm may select all of the available vector indexes, determine which vector indexes correspond to movements of an exercise being performed, and select the vector indexes which correspond to the movements of the exercise being performed. For example, the user may begin an exercise without scanning a tag. The wearable device may receive movement data of the user from a sensor of the wearable device and execute an algorithm on the movement data. The algorithm may be a default algorithm which is executed in the absence of an algorithm selected based on its association in a database with a tag identifier. The algorithm may be executed on all vector indexes of vectors of movement data in order to determine which exercise is being performed. The algorithm may determine which exercise is being performed and select an algorithm associated in the database with the exercise. The selected algorithm may select a set of vector indexes of vectors of the movement data. In some embodiments the algorithm may select a first set of vector indexes, determine that more information is needed for determining a form of a user and/or repetitions of an exercise, and select a second set of vector indexes. The second set of vector indexes may include more vector indexes than the first set of vector indexes.

The wearable device may determine what exercise is being performed based on the movement data. The wearable device may include stored patterns of exercises. The wearable device may compare vectors of the movement data to the stored patterns and determine a match. The wearable device may determine which exercise is being performed based on the match between the vectors of the movement data and the stored patterns of exercises. In some embodiments the wearable device may compare a set of vector indexes of the movement data to a set of vector indexes of the stored patterns and determine a match. In some embodiments the stored patterns are a set of vectors over time that correspond to exercises. In some embodiments the stored patterns are a set of vector indexes over time that correspond to exercises.

The wearable device may determine a form of the user based on the movement data and the stored patterns of exercises, including stored form for one or more exercises. The wearable device may compare vectors of the movement data to a pattern of the stored patterns which corresponds to an exercise being performed. The wearable device may determine differences between the vectors of the movement data and the pattern. In some embodiments the wearable device may determine differences between selected vector indexes of the movement data and the pattern. The wearable device may determine differences between the movement data and the pattern over time and/or space. Determining a form of the user may include determining that the user needs to change an aspect of their movement and/or determining that the user needs to change a speed of the exercise being performed.

The wearable device scans a tag on exercise equipment. The wearable device identifies a tag identifier of the tag. The wearable device selects an algorithm corresponding to the tag identifier. The wearable device receives movement data of a user. The wearable device selects vector indexes from the movement data, as discussed herein. The wearable device executes the algorithm on the selected vector indexes. The wearable device may query whether one repetition of the exercise has been completed. Querying whether one repetition has been completed may be an operation of the algorithm. If one repetition of the algorithm has not been completed, the wearable device may execute the algorithm on the selected vector indexes. The wearable device may continually execute the algorithm on the selected vector indexes until one repetition of the exercise has been completed.

Once the wearable device determines that one repetition of the exercise has been completed, the wearable device increases an increment counter. The increment counter counts repetitions of the exercise. The wearable device may query whether the number of repetitions exceeds a threshold. Querying whether the number of repetitions exceeds a threshold may include comparing the increment counter to the threshold. The threshold may be based on any combination of the following: the exercise being performed, an exercise history of the user, an intensity of the exercise being performed, the movement data of the user, and a workout plan of the user. In some embodiments the wearable device may determine, based on movement data of the user, a number of repetitions the user can perform before failure and determine the threshold based on the number of repetitions the user can perform before failure.

If the number of repetitions does not exceed the threshold, the wearable device may continue to receive movement data of the user. If the number of repetitions exceeds the threshold, the wearable device may query whether the exercise being performed in the last exercise. The last exercise may be the last exercise in a workout including a series of exercises or the last exercise may be based on input of the user. If the exercise is the last exercise, the wearable device may generate a record of the repetitions of the movement data. The record may include a number of repetitions of the exercise. The record may include a form of the user, including a speed of the user. The record may include a number of sets of the exercise. The wearable device may transmit the record to a remote computing device.

If the exercise is not the last exercise, the wearable device may retrieve an identification of a next exercise. The next exercise may be the next exercise in a workout including a series of exercises or the next exercise may be based on input of the user. The wearable device may present identification of the next exercise. Presenting identification of the next exercise may include a visual and/or auditory cue of the next exercise. The wearable device may generate a record of the repetitions of the movement data as repetition movement data. The record may include a number of repetitions of the exercise. The record may include a form of the user, including a speed of the user. The record may include a number of sets of the exercise. The wearable device may transmit the record to a remote computing device.

The wearable device may detect a second tag identifier for the next exercise. In some embodiments detecting the second tag identifier for the next exercise may include scanning, by the wearable device, a second tag associated with the second tag identifier for the next exercise. In some embodiments detecting the second tag identifier for the next exercise may include identifying the next exercise, presenting identification of the next exercise, and directing the user to exercise equipment containing the second tag associated with the second tag identifier for the next exercise. The wearable device may select a second algorithm corresponding to the second tag identifier. The wearable device may, in response to selecting a second algorithm, receive second movement data of the user. The wearable device may execute the second selected algorithm using the received second movement data as input. The wearable device may determine when the user has completed a second repetition of the second exercise based on the execution of the selected second algorithm using the received second movement data. The wearable device may generate a second record including second repetition movement data. The wearable device may transmit the second record to the remote computing device. The second record may include a number of repetitions of the exercise. The second record may include a form of the user, including a speed of the user. The second record may include a number of sets of the exercise.

The wearable device includes a user interface. The user interface may be a display. The user interface may include one or more indicator lights. The user interface may include one or more icons. The one or more icons may represent any combination of the following: a number of repetitions of an exercise, an indication of whether the user should perform the exercise faster or slower, a time spent on the exercise, an indication of a next exercise, an indication of progress in a workout, and an estimated amount of calories burned. Indicating to the user whether the user should perform the exercise faster or slower may include accessing a stored speed associated with an exercise, comparing a speed of the user to the stored speed, and indicating, on the user interface, at least one of the stored speed, the speed of the user, a difference between the stored speed and the speed of the user, and an indication of whether the speed of the user is higher or lower than the stored speed. The speed of the user may be determined using the movement data of the user. The absolute velocity of the wearable device may correspond to the speed of the user. The speed of the user may be compared to the stored speed for different portions of an exercise. For example, during bicep curls, the speed of the user, or the absolute value of the upward velocity of the wearable device, may be compared to a first stored speed as the user lifts a dumbbell. The speed of the user, or the absolute value of the downward velocity of the wearable device, may be compared to a second stored speed as the user lowers the dumbbell.

The user interface may include one or more user input interfaces. The user input interfaces may be buttons, switches, or areas on a display. User input may include an alteration to the sensitivity of one or more algorithms executed by the wearable device. Altering the sensitivity of the algorithms may include altering one or more weights associated with values in the movement data of the user. For example, a user may alter the sensitivity of an algorithm to multiply indexes of vectors of the movement data corresponding to movement along a vertical axis. The selected indexes of the vectors may be multiplied by a weight as the algorithm is executed on the selected indexes. This may allow the user to tune the sensitivity of the algorithm.

The wearable device may prompt a user, using a visual, auditory, and/or tactile signal, to perform an exercise. The user interface may include means for vibrating the wearable device in order to provide a tactile signal and a speaker in order to provide an auditory signal. In some embodiments the wearable device may prompt the user to perform the exercise in order to signal to the user that the wearable device is ready to receive movement data. In some embodiments the wearable device may prompt the user to perform the exercise based on a determination that a rest time has elapsed. In some embodiments the wearable device may prompt the user to perform a next exercise.

The wearable device may communicate with a mobile device in order to display the user interface of the wearable device on a display of the mobile device. In some embodiments the wearable device may send a signal to the mobile device causing the mobile device to prompt the user, using a visual and/or auditory signal, to perform the exercise. In some embodiments the mobile device may, on a display of the mobile device, display the user interface of the wearable device in order to interact with the wearable device using the mobile device. In some embodiments the user may control the wearable device using the mobile device.

A. A system comprising:

a wearable device comprising one or more processors wherein the one or more processors are configured to:
scan a tag of a set of tags on a set of weights;
identify a tag identifier of the tag;
access, from a memory of the wearable device, an algorithm associated with the tag identifier and an exercise to be performed;
receive, by a sensor of the wearable device, movement data of a user;
execute the algorithm on the movement data to determine a number of repetitions of the exercise; and
transmit the number of repetitions to a remote computing device.

B. A system comprising:

a wearable device comprising one or more processors wherein the one or more processors are configured to:
scan a tag of one or more tags on or near exercise equipment, each of the one or more tags associated with an exercise or a series of exercises;
identify a tag identifier of the tag;
determine that the tag identifier is associated with a series of exercises;
access, from a memory of the wearable device, a set of algorithms associated with the tag identifier wherein each algorithm of the set of algorithms is associated with an exercise of the series of exercises;
receive, by a sensor of the wearable device, first movement data of a user performing a first exercise of the series of exercises;
execute a first algorithm of the set of algorithms on the first movement data to determine a number of repetitions of the first exercise;
compare the number of repetitions of the first exercise to a first target number of repetitions;
determine that the number of repetitions of the first exercise is greater than or equal to the first target number of repetitions;
prompt the user, at a display of the wearable device, to proceed to a second exercise of the series of exercises;
receive, by a sensor of the wearable device, second movement data of a user performing a second exercise of the series of exercises;
execute a second algorithm of the set of algorithms on the second movement data to determine a number of repetitions of the second exercise;
compare the number of repetitions of the second exercise to a second target number of repetitions;
determine that the number of repetitions of the second exercise is greater than or equal to the second target number of repetitions; and
transmit the number of repetitions of the first and second exercises to a remote computing device.

C. The system of A or B wherein multiple tags are located on or near the exercise equipment and wherein each tag is associated with a different exercise or series of exercises to be performed using the exercise equipment.

D. A system comprising:

a tag on or near exercise equipment comprising one or more sensors, the tag being associated with an exercise or a series of exercises;

a wearable device comprising one or more processors wherein the one or more processors are configured to:
scan the tag;
identify a tag identifier of the tag;
access, from a memory of the wearable device, an algorithm associated with the tag identifier and an exercise to be performed;
receive, by a sensor of the wearable device, movement data of a user;
execute the algorithm on the movement data to determine position data of the user; and
transmit the position data to the remote computing device.

E. The system of any of A-D wherein the wearable device receives performance metrics from the one or more sensors of the exercise equipment, aggregates the performance metrics and the position data, and transmits the aggregated data to the remote computing device.

F. A system comprising:

a tag on or near exercise equipment comprising one or more sensors, the tag being associated with an exercise or a series of exercises;

a wearable device comprising one or more processors wherein the one or more processors are configured to:
scan the tag;
identify a tag identifier of the tag;
access, from a memory of the wearable device, an algorithm associated with the tag identifier and an exercise to be performed;
receive, by a sensor of the wearable device, movement data of a user;
execute the algorithm of the set of algorithms on the movement data to determine a number of repetitions of the exercise; and
transmit the number of repetitions to a remote computing device.

G. The system of any of A-F wherein the wearable device receives performance metrics from the one or more sensors of the exercise equipment, aggregates the performance metrics and the number of repetitions, and transmits the aggregated data to the remote computing device.

H. A method comprising:

detecting, by one or more processors of a wearable device, a tag identifier of a tag located on or near exercise equipment, the tag being associated with an exercise or a series of exercises;

prompting a user, by a visual and/or audible signal, to move the wearable device from a first location on a body of the user to a second position on the body of the user;

selecting, by the one or more processors from a memory of the wearable device, a first algorithm from a plurality of stored algorithms responsive to the first algorithm having a stored association with the tag identifier wherein the first algorithm is configured to receive input at the second location;

receiving, by the one or more processors from a sensor of the wearable device, first movement data of a user performing an exercise associated with the exercise equipment;

executing, by the one or more processors, the first algorithm using the received movement data as input;

determining, based on the first movement data and/or the first algorithm, that the wearable device is not located at the second location;

selecting, by the one or more processors from the memory of the wearable device, a second algorithm from a plurality of stored algorithms responsive to the second algorithm having a stored association with the tag identifier wherein the second algorithm is configured to receive input at the first location;

receiving, by the one or more processors from the sensor of the wearable device, second movement data of a user performing the exercise associated with the exercise equipment;

executing, by the one or more processors, the second algorithm using the second movement data as input;

determining, by the one or more processors, when the user has completed one repetition of the exercise based on the execution of the second algorithm using the second movement data;

and transmitting, by the one or more processors, a record comprising repetition movement data that was received during the one repetition to a remote computing device.

I. The method of H wherein determining that the wearable device is not located at the second location comprises determining, by the one or more processors, that a user input has not been received within a defined time period after prompting the user to move the wearable device from the first location on a body of the user to the second position on the body of the user.

J. The method of H or I wherein determining that the wearable device is not located at the second location comprises receiving a user input indicating that the wearable device has not been moved from the first location to the second location in response to prompting the user to move the wearable device from the first location on a body of the user to the second position on the body of the user.

K. A system comprising:

a tag on or near exercise equipment, the tag being associated with an exercise or a series of exercises;

a wearable device comprising one or more processors wherein the one or more processors are configured to:
scan the tag;
identify a tag identifier of the tag;
access, from a memory of the wearable device, an algorithm associated with the tag identifier and an exercise to be performed;
receive, by a sensor of the wearable device, movement data of a user;
execute the algorithm of the set of algorithms on the movement data to determine a number of repetitions of the exercise; and
transmit the number of repetitions to a remote computing device.

L. The system of any of A-G or K wherein the one or more processors of the wearable device are further configured to transmit at least one of raw movement data, a set of vectors of the movement data, and a set of vector indexes of the movement data.

M. The system of any of A-G and K-L further comprising a second wearable device comprising one or more processors configured to:

access, from a second memory of the second wearable device, a second algorithm associated with the tag identifier and the exercise to be performed;

receive, from a second sensor of the second wearable device, second movement data of a user;

execute the second algorithm on the second movement data to determine a second number of repetitions of the exercise; and

transmit the second number of repetitions of the exercise to the remote computing device;

N. The system of any of A-G and K-M wherein transmitting the second number of repetitions of the exercise to the remote computing device comprises:

selecting one of the wearable device and second wearable device by the one or more processors of the first and second wearable devices;

compiling the first and second numbers of repetitions of the exercise at the selected wearable device by the one or more processors of the selected wearable device; and

transmitting the first and second numbers of repetitions of the exercise to the remote computing device.

O. The system of any of A-G and K-N wherein the one or more processors of the wearable device are further configured to:

increment a counter of a number of sets of the exercise;

record a record of a number of repetitions of the exercise performed in each set; and

transmit the number of sets and the record of the number of repetitions of the exercise performed in each set to the remote computing device.

P. The system of any of A-G and K-O wherein the one or more processors of the wearable device are further configured to:

calculate, based on the number of repetitions of the exercise, an estimated calorie burn;

and transmit the estimated calorie burn to the remote computing device.

Q. A method comprising:

scanning, by one or more processors of a wearable device, a tag located on or near exercise equipment;

identifying, by the one or more processors of the wearable device, a tag identifier of the tag;

transmitting, by the one or more processors of the wearable device, the tag identifier to a remote computing device;

in response to receiving the tag identifier from the wearable device, accessing, by one or more processors of the remote computing device, from a memory of the remote computing device, an algorithm associated with the tag identifier and an exercise;

transmitting the algorithm, by the one or more processors of the remote computing device, to the wearable device;

receiving, by one or more sensors of the wearable device, movement data of a user;

executing, by the one or more processors of the wearable device, the algorithm on the movement data to determine a number of repetitions of the exercise;

generating, by the one or more processors of the wearable device, a record of the number of repetitions of the exercise; and

transmitting, by the one or more processors of the wearable device, the record of the number of repetitions of the exercise to the remote computing device.

R. The system of any of A-G and K-P wherein the wearable device is located on a chest of a user.

S. The system of any of A-G and K-P wherein the wearable device is located on a wrist of a user.

T. The system of any of A-G and K-P wherein the wearable device is located on a thigh of a user.

U. The system of any of A-G and K-P wherein the wearable device is located on a hand of a user.

V. The system of any of A-G and K-P wherein the wearable device is located on a hip of a user.

W. The system of any of M-P wherein the second wearable device is located on a chest of a user.

X. The system of any of M-P wherein the second wearable device is located on a wrist of a user.

Y. The system of any of M-P wherein the second wearable device is located on a thigh of a user.

Z. The system of any of M-P wherein the second wearable device is located on a hand of a user.

AA. The system of any of M-P wherein the second wearable device is located on a hip of a user.

BB. The method of any of H-J and Q wherein the wearable device is located on a chest of a user.

CC. The method of any of H-J and Q wherein the wearable device is located on a wrist of a user.

DD. The method of any of H-J and Q wherein the wearable device is located on a thigh of a user.

EE. The method of any of H-J and Q wherein the wearable device is located on a hand of a user.

FF. The method of any of H-J and Q wherein the wearable device is located on a hip of a user.

GG. The system of any of A-G and K-P wherein the sensor is located on a finger of the wearable device.

HH. The system of any of A-G and K-P wherein the sensor is located on a thumb of the wearable device.

II. The system of any of A-G and K-P wherein the sensor is located on a wrist of the wearable device.

JJ. The system of any of A-G and K-P wherein the sensor is located on a back portion of the wearable device.

KK. The method of any of H-J and Q wherein the sensor is located on a finger of the wearable device.

LL. The method of any of H-J and Q wherein the sensor is located on a thumb of the wearable device.

MM. The method of any of H-J and Q wherein the sensor is located on a wrist of the wearable device.

NN. The method of any of H-J and Q wherein the sensor is located on a wrist of the wearable device.

OO. The method of any of H-J and Q wherein the sensor is located on a back portion of the wearable device.

In an illustrative embodiment, any of the operations described herein can be implemented at least in part as computer-readable instructions stored on a computer-readable medium or memory. Upon execution of the computer-readable instructions by a processor, the computer-readable instructions can cause a computing device to perform the operations.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A method comprising:
detecting, by one or more processors of a wearable device, a tag identifier of a tag located on exercise equipment;
selecting, by the one or more processors from a memory of the wearable device, an algorithm from a plurality of stored algorithms responsive to the algorithm having a stored association with the tag identifier;
receiving, by the one or more processors from a sensor of the wearable device, movement data of a user performing an exercise associated with the exercise equipment;
executing, by the one or more processors, the selected algorithm using the received movement data as input;
determining, by the one or more processors, when the user has completed one repetition of the exercise based on the execution of the selected algorithm using the received movement data; and
transmitting, by the one or more processors, a record comprising repetition movement data that was received during the one repetition to a remote computing device.

2. The method of claim 1, wherein executing the selected algorithm comprises:
selecting, by the one or more processors, an index of a gyroscopic vector from the received movement data based on the tag identifier or the selected algorithm; and
executing, by the one or more processors, the selected algorithm using only the selected index of the movement data as input.

3. The method of claim 1, wherein executing the selected algorithm comprises determining, by the one or more processors, one or more performance parameters of the user performing the exercise, and further comprising:
transmitting, by the one or more processors, the one or more performance parameters to the remote computing device.

4. The method of claim 1, wherein determining when the user has completed one repetition comprises:
comparing, by the one or more processors, consecutive vectors of the received movement data to identify a difference in values between the consecutive vectors; and
determining, by the one or more processors, the user has completed one repetition based on the difference satisfying a repetition policy.

5. The method of claim 1, wherein executing the selected algorithm by the one or more processors comprises:
determining, by the one or more processors, a speed of the user from the received movement data;
comparing, by the one or more processors, the speed to a stored speed associated with the selected algorithm; and
further comprising notifying, by the one or more processors, the user, on a display of the wearable device, of the stored speed, the speed of the user, or a difference between the stored speed and the speed of the user.

6. The method of claim 1, further comprising prompting the user, by the one or more processors, using an audible and/or visual signal, to perform the exercise.

7. The method of claim 1, wherein executing the selected algorithm comprises identifying, by the one or more processors, the exercise performed by the user based on the movement data and/or the tag identifier.

8. The method of claim 1, further comprising:
receiving, by the one or more processors, input from the user, receiving the input causing the one or more processors to alter one or more weights associated with values in the user movement data when the one or more processors execute the selected algorithm.

9. The method of claim 1, further comprising prompting the user, by the one or more processors, using an audible and/or visual signal, to place the wearable device on a body part of the user.

10. The method of claim 1, further comprising:
maintaining, by the one or more processors, a counter indicating a number of repetitions the user has performed of the exercise;
incrementing, by the one or more processors, the counter responsive to the determining the user has completed the one repetition based on the execution of the selected algorithm using the received movement data; and
prompting the user, by the one or more processors, using an audible and/or visual signal, to stop the exercise and begin a second exercise responsive to a count of the counter exceeding a predetermined threshold. based on a count of repetitions of the exercise.

11. The method of claim 1, further comprising:
detecting, by the one or more processors of the wearable device, a second tag identifier of a second tag located on second exercise equipment;
selecting, by the one or more processors from the memory of the wearable device, a second algorithm from the plurality of stored algorithms responsive to the second algorithm having a second stored association with the second tag identifier;
receiving, by the one or more processors from the sensor of the wearable device, second movement data of the user performing the second exercise;
executing, by the one or more processors, the second selected algorithm using the received second movement data as input;
determining, by the one or more processors, when the user has completed a second repetition of the second exercise based on the execution of the selected second algorithm using the received second movement data; and
transmitting, by the one or more processors, a second record comprising second repetition movement data that was received during the second repetition to the remote computing device.

12. The method of claim 1, wherein executing the selected algorithm comprises determining, by the one or more processors, a form of the user performing the exercise from the user movement data and comparing the form of the user to a stored form.

13. The method of claim 1, wherein receiving movement data of the user comprises receiving movement data from a second sensor of a second wearable device and wherein executing the selected algorithm using the one or more processors includes aggregating data from the sensor of the wearable device and the second sensor of the second wearable device.

14. A system comprising:
a wearable device comprising one or more processors, a memory, a plurality of communications interfaces, and one or more sensors, wherein the one or more processors:
detect a tag identifier of a tag located on exercise equipment;
select, from the memory of the wearable device, an algorithm from a plurality of stored algorithms responsive to the algorithm having a stored association with the tag identifier;
receive, from a sensor of the one or more sensors via a first communications interface of the plurality of communications interfaces, movement data of a user performing an exercise associated with the exercise equipment;
execute the selected algorithm using the received movement data as input;
determine when the user has completed one repetition of the exercise based on the execution of the selected algorithm using the received movement data; and
transmit, via a second communications interface of the plurality of communications interfaces, a record comprising repetition movement data that was received during the one repetition to a remote computing device.

15. The system of claim 14, wherein the one or more processors execute the selected algorithm by:
select an index of a gyroscopic vector from the received movement data based on the tag identifier or the selected algorithm; and
execute the selected algorithm using only the selected index of the movement data as input.

16. The system of claim 14, wherein the one or more processors execute the selected algorithm by determining one or more performance parameters of the user performing the exercise, and wherein the one or more processors further:
transmit the one or more performance parameters to the remote computing device.

17. The system of claim 14, wherein the one or more processors determine when the user has completed one repetition by:
comparing consecutive vectors of the received movement data to identify a difference in values between the consecutive vectors; and
determining the user has completed one repetition based on the difference satisfying a repetition policy.

18. The system of claim 14, wherein the one or more processors execute the selected algorithm by:
determining a speed of the user from the received movement data; and
comparing the speed to a stored speed associated with the selected algorithm; and
wherein the one or more processors further:
notify the user, on a display of the wearable device, of the stored speed, the speed of the user, or a difference between the stored speed and the speed of the user.

19. The system of claim 14, wherein the one or more processors further prompt the user using an audible and/or visual signal, to perform the exercise.

20. The system of claim 14, wherein the one or more processors execute the selected algorithm by identifying the exercise performed by the user based on the movement data and/or the tag identifier.
